(19) United States
(12) Patent Application Publication
(10) Pub. No.: US 2024/0093262 A1
(43) Pub. Date: Mar. 21, 2024

(54) SYSTEM AND METHOD OF EXPLOITING MICROBIAL METABOLIC PROCESSES FOR USE AS A BIOSENSOR IN WATER QUALITY MONITORING AND OTHER APPLICATIONS

(71) Applicant: AquaSignum Inc., Toronto (CA)

(72) Inventors: Spencer Ronald CROOK, Toronto (CA); Romeo Gabriel DUMITRACHE, East York (CA); Marthinus KROUKAMP, Mississauga (CA); Evan Lindsay Gilmore RONAN, Toronto (CA); Patrick Charles Gilmore RONAN, Toronto (CA)

(73) Assignee: AquaSignum Inc., Toronto, ON (CA)

(21) Appl. No.: 17/754,856

(22) PCT Filed: Oct. 15, 2020
(86) PCT No.: PCT/CA2020/051383

§ 371 Date: Apr. 14, 2022

Related U.S. Application Data
(60) Provisional application No. 62/915,675, filed on Oct. 16, 2019.

(51) Int. Cl. C12Q 1/04

(52) U.S. Cl. CPC C12Q 1/04

(57) ABSTRACT

Embodiments described herein relate to a system, method, and sensors for real-time microbial monitoring based on the presence and concentrations of microbial signals, typically gaseous compounds, which are reflective of the microbial population size, microbial health, and/or microbial metabolic activity level within aqueous environments. Use of the disclosed technology to provide online remote measurement of microbial signals, and importantly the detection of changes therein, can be used to determine stable operating conditions and detect fluctuations in water quality. The sensor monitoring technology is able to monitor the native microbial population present in an aquatic environment and does not consume any reagents or require discrete sampling points. Further, an online measurement can be implemented to track microbial activity in real-time.

### CROSS REFERENCE TO RELATED APPLICATIONS

The application is a National Phase application that claims priority to and the benefit of the international PCT Patent Application No. PCT/CA2020/051383 entitled “SYSTEM AND METHOD OF MONITORING MICROBIAL METABOLIC PROCESSES”, filed on Oct. 15, 2020, which claims priority to and the benefit of U.S. Provisional Patent Application Ser. No. 62/915,675, entitled “SYSTEM AND METHOD OF EXPLOITING MICROBIAL PROCESSES FOR WATER QUALITY MONITORING”, filed on Oct. 16, 2019, the disclosure of which are incorporated herein by reference in their entirety.

### FIELD OF INVENTION

Embodiments described herein relate to systems, methods, and sensors for monitoring microbial population size, microbial health, and/or microbial metabolic activity levels, and importantly, changes therein. The disclosed invention can be used as a biosensor for real-time monitoring of water quality in natural and engineered water and wastewater systems, in addition to other environmentally and industrially relevant applications.

### BACKGROUND

Water quality in natural and engineered water and wastewater systems is dynamic and can vary based on a multitude of factors such as changing environmental, chemical, physical, and biological conditions. For example, the quality of surface and ground water can change due to natural processes such as rock weathering, soil leaching, evapotranspiration, deposition by wind, run-off caused by hydrological factors, as well as changing biological processes occurring in the water. These natural water systems can also deteriorate rapidly due to anthropogenic factors such as nutrient run-off from agricultural and other lands, as well as sewage infiltration caused by sewer overflows, cross-connections in the sewage infrastructure, or illegal dumping. In each case, changes in the quality of surface and ground water can have negative effects such as increasing the risk to public health, and so methods and systems to rapidly detect such changes are needed.

Engineered systems such as wastewater treatment systems, drinking water treatment and distribution systems, water-cooling towers, and many other industrial processing systems are also vulnerable to changing water quality due to factors such as the presence of toxins, chemical shock loads, as well as biofouling and scaling. In wastewater treatment, unexpected changes in water quality can lead to system upsets which may increase energy demands, reduce treatment efficacy, and result in the discharge of insufficiently treated effluent to receiving waters. In drinking water systems, changes in water quality can impair treatment and disinfection processes resulting in non-potable water that presents a threat to public health. In cooling towers, increased microbial growth can impair system processes such as efficient heat transfer, leading to increased use of energy and costly biocides. Furthermore, there are countless industrial processes which rely on stable water quality, and so unexpected changes in this quality can lead to significant problems such as interruptions to production lines. Similarly, pilot and industrial-scale digesters, fermenters, and bioreactors which depend on healthy, active and stable microbial populations and/or microbial metabolic activity can be impaired by system upsets arising from changes in physical or chemical parameters. This can greatly hinder the efficiency of these processes or halt them altogether, resulting in downtime or inefficient production of end products. In all cases, whether in natural or engineered systems, failure to promptly recognize when water quality is changing can delay the implementation of necessary remedial actions and cause a multitude of negative environmental, economic, and social impacts.

Since microbes react very quickly to changes in their environment, microbial monitoring can be used to monitor the stability of environmental and industrial processes and to provide the earliest detection of changes which could include changing water quality in natural and engineered water and wastewater systems. However, current technologies which seek to monitor water quality through microbial monitoring are hindered by several issues. First, any culture-based technique cannot achieve real-time data generation and does not accurately account for the presence and activity of viable but non-culturable microbes in the systems being monitored. As well, most current approaches to microbial monitoring cannot provide online and continuous insight into water quality and instead require discrete sampling points and rely on consumable reagents. Probe-based water quality monitoring techniques are also often adversely affected by environmental contaminants which can amplify or quench the signal being measured. These competing technologies are also usually hindered by biofouling, whereby the formation of a biofilm on the measuring device leads to impaired function and in nutrient rich environments with robust growth microbial populations, routine cleanings or full replacement of devices are required to maintain accurate sensing. Thus, there is a need for technologies which can overcome these limitations to reliably and remotely achieve microbial monitoring.

Continuous monitoring of microbial population size, microbial health, and/or microbial metabolic activity levels can be achieved by detecting and measuring the presence and concentration of microbial signals such as gaseous compounds that are produced and/or consumed during microbial growth and/or used as a metabolite during microbial metabolic activity. Real-time monitoring of these microbial signals can be used to provide unique insights into the stability and quality of natural and engineered water and wastewater systems, and importantly, facilitate the rapid detection of changes therein.

### SUMMARY

Embodiments described herein relate to systems, methods, and sensors for monitoring microbial population size, microbial health, and microbial metabolic activity levels, and importantly, changes therein. The present disclosure relates to a biosensor technology that facilitates real-time, online, and remote measurement of a microbial signal that is typically but not exclusively one or more gaseous compounds (herein referred to as “microbial signals”), whose presence, production and/or consumption is reflective of the microbial population size, microbial health, and/or microbial metabolic activity level within aqueous environments. Briefly, the biosensor technology involves the use of membranes with permeability to one or more microbial signals of interest, and which are oriented in a manner that creates a gaseous cavity into or out of which microbial signals may diffuse but bulk water is excluded. This allows for microbial signals originating from microbes growing on or near the membranes to be collected, transported and measured using suitable sensors, analyzers, and/or detectors, ultimately to determine the presence and concentration of each microbial signal of interest. One embodiment of the disclosed technology involves a “biomass scaffold” which incorporates a gas permeable tubular membrane that collects microbial signals and carries them via forced flow of a sweeper gas to a “central hub”, wherein the signals are measured, logged, analyzed, displayed, and/or transmitted to the internet or other network. The biosensor technology can be used to monitor the population size, health, and/or metabolic activity level of the native microbial population present in any natural or engineered system, or it may be used to monitor specific or desired microbial populations though pre-treatment and/or pre-colonization of the membranes. It is also able to provide microbial monitoring under a very wide range of environmental conditions, including those which are aerobic, anoxic, and anaerobic, partly due to the ability to detect and measure one or more microbial signals simultaneously, as well as by allowing for the ability to dial and/or tune the relative strength of each microbial signal via simple adjustments such as to the surface area of the membrane and/or the flow rate of the sweeper gas. The biosensor technology represents an improvement on the state of the art, as it is not hindered by biofouling, does not consume any reagents, and does not require discrete sampling points. In addition to the design and function of the disclosed biosensor technology, significant novelty lies in the use of this system for providing environmentally and industrially relevant information, such as in applications to provide real-time monitoring of water quality in natural and engineered water and wastewater systems.

### BRIEF DESCRIPTION OF THE DRAWINGS

FIG. 1A is a schematic diagram illustrating the base system, a CO₂-evolution measurement system (CEMS).

FIG. 1B is a cross section diagram of the base system, illustrating radial transfer of CO₂ from the liquid bulk phase to the gas bulk phase.

FIG. 2 is a graph that depicts rapid microbial response to controlled manipulation of growth temperature.

FIG. 3A are graphs illustrating rapid and linear microbial responses to controlled manipulation of the concentrations of available nutrients.

FIG. 3B are graphs illustrating rapid and sensitive microbial responses to controlled manipulation of carbon availability under both aerobic and anoxic growth conditions in which the terminal electron acceptor used by the microbial metabolism could change.

FIG. 3C is a correlation plot showing linearity in the relationship between carbon availability and microbial signals.

FIG. 4A are graphs showing sensitive and rapid microbial metabolic responses to the presence of streptomycin antibiotic under differing oxygen and nutrient conditions.

FIG. 4B is graph showing sensitive and rapid microbial metabolic response to antibiotic exposure.

FIG. 5 is a graph showing sensitive and rapid microbial response to changes in oxygen availability.

FIG. 6A is a graph showing microalgae activity, given as the rate of net CO₂ consumed per hour, under alternating twelve-hour light-dark cycles.

FIG. 6B is a graph showing microalgae activity, given as the rate of net CO₂ consumed per hour, during changing light intensity.

FIG. 7 is a schematic depicting a comprehensive modification of the base system whereby a permeable tubular membrane is used to grow biomass (e.g. biofilms) on the outer surface of the tube.

FIG. 8 is a diagram showing two iterations of a biomass scaffold incorporating a permeable tubular membrane prior to being colonized with microbial biomass.

FIG. 9 is a diagram showing two iterations of the biomass scaffold incorporating a permeable tubular membrane after being colonized with microbial biomass.

FIG. 10 is a diagram showing two iterations of the biomass scaffold with different protective casings around the scaffold.

FIG. 11A is a front view of the central hub.

FIG. 11B is a right side view of the central hub.

FIG. 11C is a left side view of the central hub.

FIG. 11D is a rear view of the central hub.

FIG. 11E is diagram illustrating direction of gas flow.

FIG. 11F is a diagram of the central hub with the lid open.

FIG. 11G is a diagram of the central hub with the lid lifted.

FIG. 11H is a diagram of the central hub illustrating access of the internal pump.

FIG. 12 is a screenshot of one embodiment of the software as displayed on the LCD touchscreen of the central hub

FIG. 13 is a diagram showing one embodiment of a user-facing online dashboard used to display microbial activity data.

FIG. 14 is a schematic diagram showing one possible application of one embodiment of the disclosed technology for monitoring environmental sewage infiltration.

FIG. 15 is a schematic depicting the process steps of a generic wastewater treatment system and indicating where the disclosed technology could be used.

FIG. 16A is a graph illustrating microbial signal in response to simulated sewage spikes.

FIG. 16B are graphs showing individual responses to sewage spikes in terms of CO₂ production values.

FIG. 16C is a chart that shows analysis of the data related to microbial responses to sewage spikes.

FIG. 17 are graphs showing the change in microbial response to sewage spike when the length of the tubular membrane was doubled.

FIG. 18 is a graph illustrating the microbial response to fertilizer in an aqueous environment.

FIG. 19 is a graph showing the microbial response when a membrane-bound biofilm was exposed to sewage after the biofilm had been dried out for many weeks.

FIG. 20 is a graph showing real-world data collected by one embodiment of the technology when used to monitor the influent stream of a wastewater treatment plant.

FIG. 21 is a graph showing real-world data collected by one embodiment of the technology when used to monitor a natural stream system.

FIG. 22 is a schematic diagram showing the “open-loop” configuration of the technology whereby the sweeper gas flows in a once-through linear path.

FIG. 23A is a schematic diagram showing the “closed-loop” configuration of the technology whereby the sweeper gas is cycled continuously through the system.

FIG. 23B is a schematic diagram showing the use of valves to interchange between the “closed-loop” and “open-loop” configurations of the technology.

### DETAILED DESCRIPTION

There are many chemical and physical parameters which can affect microbial population size, microbial health, and/or microbial metabolic activity levels. Previous use of a base system known as a CO₂ evolution measurement system (CEMS) as a laboratory-based research tool in a range of scientific studies has demonstrated the usefulness of measuring microbial signals, in this case CO₂, to determine how microbes respond to artificial and controlled manipulation of their growth conditions. The present disclosure covers systems, methods, and sensors which represent a novel and non-obvious advancement of the base system, whereby continuous measurement of microbial signals, including but not limited to gaseous compounds such as CO₂, CO, O₂, O₃, H₂, H₂S, CH₄, SO₂, N₂, NO₂, NO, N₂O etc., can be used to monitor the quality and stability of natural and engineered systems where strict control over chemical and physical parameters is usually not possible. For example, one important area of application covered by the present disclosure is for the continuous and remote monitoring of water quality in natural and engineered water and wastewater systems, and importantly, the rapid detection of changes therein. This is because when unrecognized chemical and physical changes occur in these systems due to natural and/or anthropogenic factors, they can have detrimental environmental, economic, and/or social impacts.

The conventional approach to water quality monitoring is usually to measure individual chemical and physical parameters, either continuously using probes or based on discrete points of sampling and analyses. There are no technologies that can simultaneously monitor and detect changes in all the countless chemical and physical parameters that can affect water quality through individual or interaction effects. However, continuous monitoring of microbial signals whose presence and concentrations are reflective of microbial population size, microbial health status, and/or microbial metabolic activity levels represents a holistic approach to water quality monitoring, since microbes react extremely quickly (detectable within seconds or minutes) to both chemical and physical changes in their environment. Real-time monitoring of microbial signals can therefore provide unique insights into the stability of natural and engineered systems, and importantly, offer a quick and cost-effective means to detect changes in water quality therein, and allow for rapid and responsive implementation of preventative or remedial actions when necessary.

Four general scenarios have been identified in which continuous monitoring of microbial signals can be used to track the microbial population size, microbial health, and/or microbial metabolic activity levels to provide unique and important insights into water quality in natural and engineered systems.
i. Continuous monitoring of microbial signals can be used to elucidate the stable, optimal, and/or desired operating conditions in natural and engineered systems, and to ensure the continued stability of said systems. “Stability” in microbial signals may take the form of regular and repeating patterns or cycles, such as diurnal changes. Elucidating stable, optimal, and/or desired operating conditions with respect to microbial signals allows for the ability to compare between similar systems.
ii. Transition from a state of no or very low level of microbial signals to a state with higher and possibly increasing level of microbial signals. This change (herein referred to as “biofouling”) may be in response to intentional or unintentional physical and/or chemical changes in natural or engineered systems. This could include but is not limited to increasing concentrations of chemicals which are beneficial for microbial growth and/or decreasing concentrations and/or efficacy of antimicrobials, biocides, disinfectants, etc. that are meant to control or prevent microbial growth.
iii. Transition from a state of stable, predictable, and/or desired level of microbial signals to a state with lower and possibly decreasing level of microbial signals. This change may be in response to intentional or unintentional physical and/or chemical changes in water. This could include but is not limited to introduction of inhibitory chemicals such as toxic compounds, antimicrobials, biocides, disinfectants, etc. and/or actions which lead to physical disruption and/or removal of microbes from the system. It could also be the result of high and possibly increasing consumption of microbial signals by the microbes being monitored, such as in the consumption of CO₂ by autotrophic microbes.
iv. Transition from a state of stable, predictable, and/or desired level of microbial signals to a state with higher and possibly increasing level of microbial signals. This change may be in response to intentional or unintentional physical and/or chemical changes in water. This could include but is not limited to increasing concentrations of chemicals which are beneficial for microbial growth and/or decreasing concentrations and/or efficacy of antimicrobials, biocides, disinfectants, etc. that are meant to control or prevent microbial growth.

Each of the above general scenarios can occur readily in natural and engineered systems, and many use cases have been identified wherein the disclosed technology can provide real-time insight into the stability of these systems. Continuous monitoring of microbial signals can be used to ensure continued stability of systems, to optimize system processes or performance, or to provide early detection of changing chemical and/or physical parameters. A major advantage of the disclosed technology is the ability to monitor microbial signals under a very wide range of environmental conditions, including those which are aerobic, anoxic, and/or anaerobic. That is, microbial monitoring is possible under variable redox states wherein microbes may use one or more different terminal electron acceptors for their metabolic processes.

There are many possible applications in which microbial monitoring provided by the disclosed technology can offer value and be used to solve environmentally and/or industrially relevant problems. Each use case has been validated through extensive research and consultation with end-users, public agencies, private corporations, regulatory bodies, and other relevant stakeholders. There is significant utility of the disclosed technology in the wastewater treatment sector, such as to monitor the stability of each treatment stage or to gauge the relative “strength” of wastewater that enters treatment systems. It can also be used to detect nutrient shock loads or toxin inflow in the influent stream, to optimize parameters such as aeration, dosing of chemicals such as carbon, alkalinity, chlorine, etc., and to monitor effluent quality to ensure adequate pollutant removal. It may also be used to provide real-time monitoring of anaerobic digestion systems, allowing for immediate feedback regarding methane production by methanogenic microbes. The biosensor may also be used as a method to provide real-time alerting of undesired microbial growth and/or when microbial metabolic activity levels exceed desired levels, such as during biofouling of drinking water collection and distribution systems, in wells and cisterns, within water cooling towers, or within ship ballast water. In addition, the biosensor may be used to provide passive environmental monitoring of surface and/or ground water to track the overall stability of these systems and/or to detect events such as nutrient infiltration (eutrophication) and algal blooms, or to detect sewage infiltration caused by combined sewer overflows, cross connections in the sewage infrastructure, or illegal dumping. It could also be used to provide monitoring within hot water tanks and hot water pipes, in order to determine if and when conditions become favourable for the proliferation of pathogenic microbes such as Legionella sp. There are also numerous industrial processes which could benefit from the application of real-time monitoring of microbial signals, such as to monitor fermentation efficiency in brewing and winemaking, to monitor microbial biofuel production, or to track the stability and detect upsets within aquaculture systems.

### Details of Underlying Technology

The present disclosure covers systems, methods, and sensors which represent a novel and non-obvious advancement of a base system known as a CO₂ evolution measurement system (CEMS). This base system was originally used to determine a complete carbon balance across microbial biofilms and was since used as a laboratory-based research tool for studying how microbes react to artificial and controlled manipulation of physical and chemical growth conditions, based on whole-biofilm CO₂ production rates. This has included measuring microbial responses to intentional changes in temperature and carbon availability, as well as during other chemical and physical manipulations (e.g. antibiotic challenges).

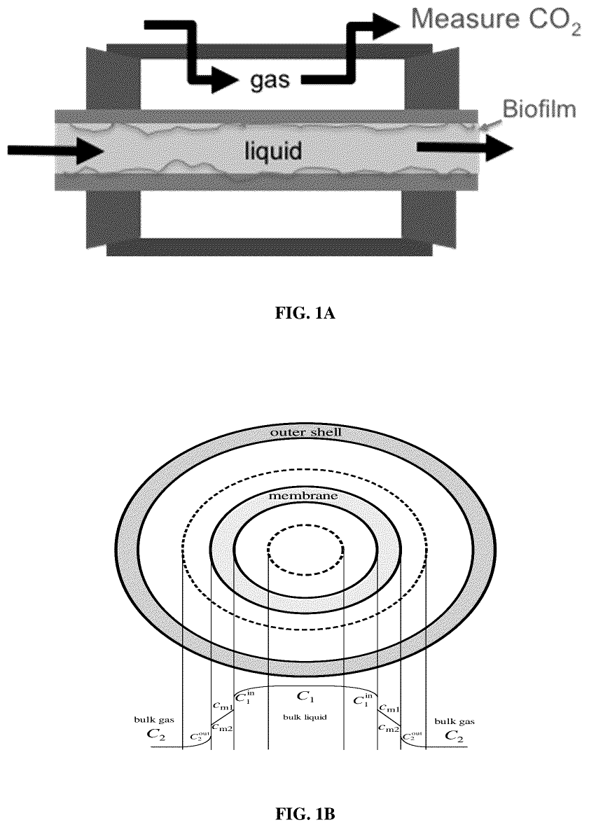

FIG. 1A is a schematic diagram illustrating the base system, a CO₂-evolution measurement system (CEMS). According to FIG. 1A, the base system consists of a silicone tube biofilm reactor encased in a sealed Tygon tube with the annular space being connected to a CO₂ analyzer. Silicone tubing has a relatively high gas permeability compared to Tygon tubing (e.g. approximately 50 times and 200 times higher for CO₂ and O₂, respectively) and so a fraction of the biofilm-produced CO₂ crosses the inner silicone tube wall into the annular space and is carried via a sweeper gas to a non-dispersive, infrared LI-820 CO₂ gas analyzer.

FIG. 1B is a cross section diagram illustrating radial transfer of CO₂ from the liquid bulk phase to the gas bulk phase in the CEMS. According to FIG. 1B, there exists a CO₂ concentration gradient between the inside of the inner tube where the biofilm grows and the annular space, and therefore the CO₂ will move along this gradient into the annular space from where it can be shuttled to an analyzer for quantification. The lines are schematic representations of the concentrations presumed to be present at steady state; e.g. within the silicone tube wall we assume a linear gradient and just adjacent to the tube wall we expect a non-linear gradient that will be dependent on the flow conditions on either side of the wall.

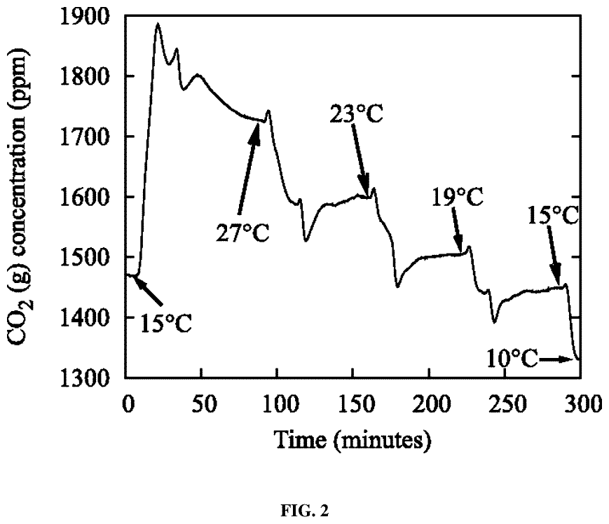

The base system proved to be a valuable tool for laboratory-based research, as it allowed for measurement of microbial responses to intentional and controlled manipulation of chemical and physical parameters affecting microbes. For example, FIG. 2 is a graph showing data obtained with the base system, which highlights the rapid microbial response to changing temperature.

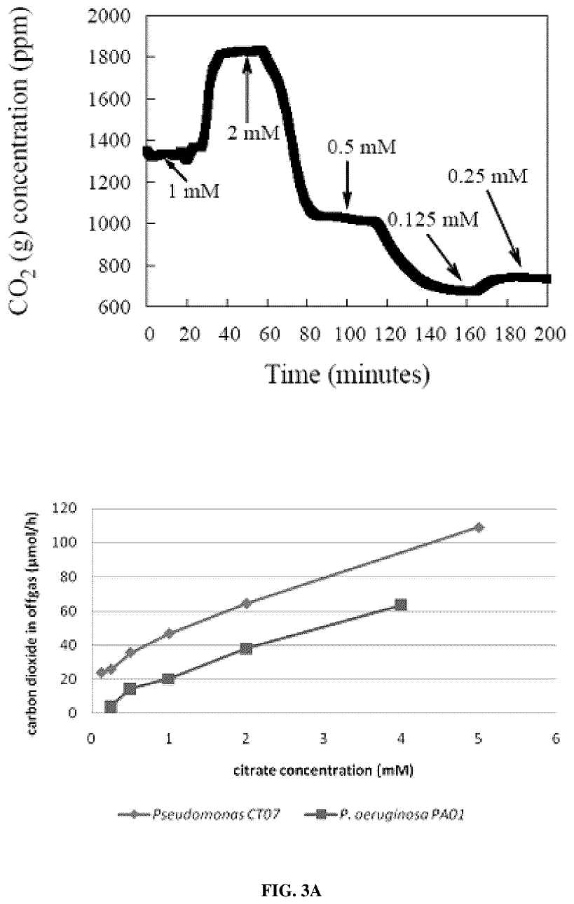

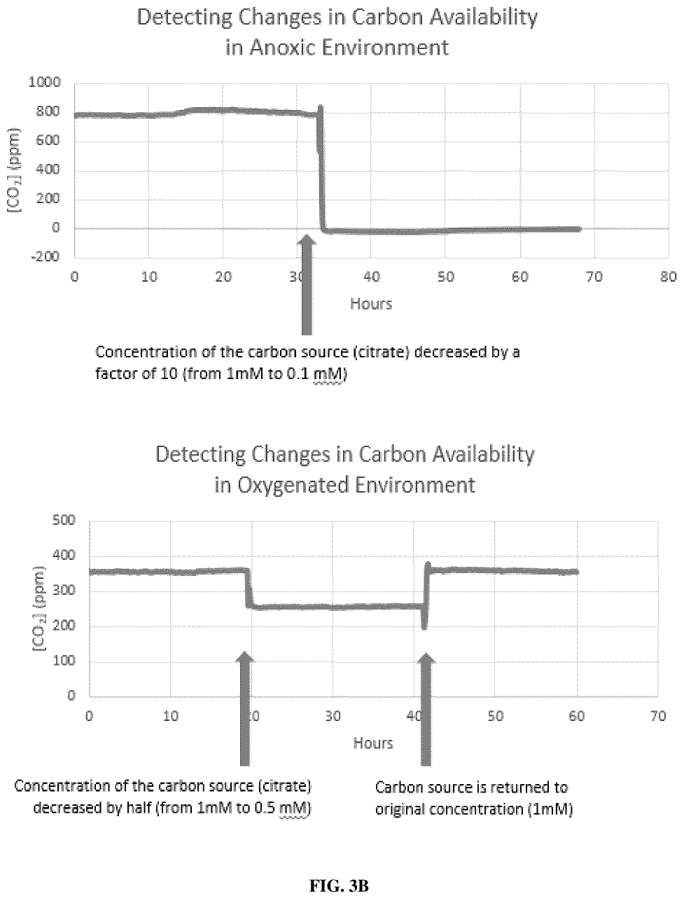

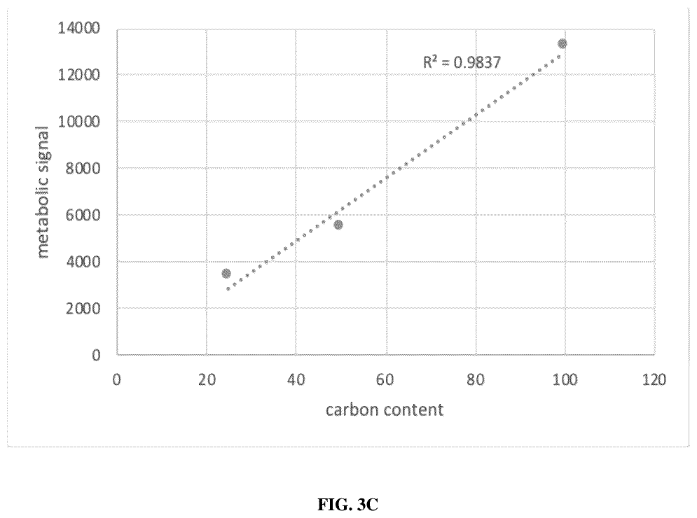

FIG. 3A are graphs illustrating rapid and linear microbial responses to changes in the concentrations of available nutrients. FIG. 3B are graphs illustrating rapid and sensitive microbial responses to changes in carbon availability under both aerobic and anoxic growth conditions in which the terminal electron acceptor used by the microbial metabolism could change. FIG. 3C is a correlation plot showing linearity in the relationship between carbon availability and microbial signals.

According to FIG. 3A, laboratory data is collected using the base system which illustrates a rapid and linear response in microbial CO₂ production rates to changes in the concentrations of available nutrients. This shows that microbial signal(s) such as CO₂ production are directly reflective of the concentration of nutrient(s) in its liquid growth medium. This is further demonstrated by the graphs in FIG. 3B, which also show sensitive and rapid microbial metabolic responses to changes in carbon availability under both aerobic and anoxic growth conditions. Overall, the laboratory-based experimentation using the base system demonstrated strong correlation between the carbon content of the growth media and the production of metabolic signals such as CO₂. This is highlighted by the correlation plot shown in FIG. 3C. This scientific insight demonstrated the potential for using of whole-biofilm metabolic signal measurements as a rapid indication of changing water quality in natural and engineered water and wastewater systems.

The original base system was also used in a number of research and laboratory-based applications, such as to measure carbon flow through the microbial biofilm food web and related carbon partitioning between biomass and the environment. These early studies clearly established the principle which underlies the present disclosure, as indicated by their publication in high-impact peer-reviewed journals. Further laboratory research utilized the base system to delineate the flow of carbon through bacterial biofilms capable of converting plant biomass to ethanol for biofuel production under anaerobic conditions. Subsequent collaboration with a leading international modeler enabled researchers to further validate these approaches, which served to highlight the potential of using microbial signals such as CO₂ to inform anaerobic processes, including in the design of anaerobic bioreactors, determining the effect of pre-treatments, and elucidating the relative importance of microbial and substrate limitation during various stages of bioprocessing.

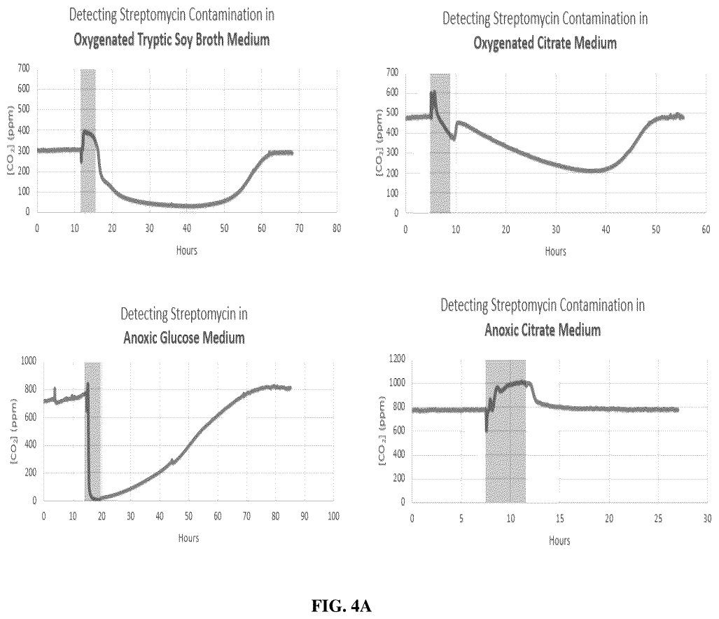

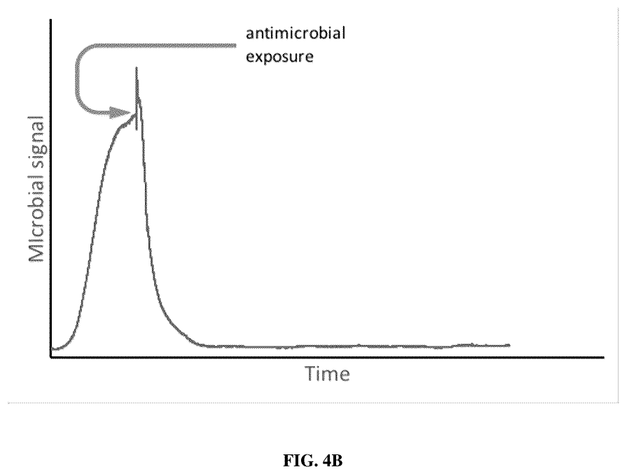

Further laboratory-based research used the base system to measure real-time microbial responses to antimicrobial exposures and combined antibiotic and ultrasonic treatment. The graphs in FIG. 4A present data collected using the base system which illustrates sensitive and rapid microbial metabolic responses to the presence of streptomycin antibiotic under differing oxygen and nutrient conditions. This shows that microbial signals such as CO₂ production are directly reflective of, and rapidly impacted by, the presence of antibiotics. Furthermore, these graphs demonstrate that microbial signals such as CO₂ production can be used to determine if and when microbial health and activity return to pre-antibiotic exposure levels once the antibiotic is no longer present.

FIG. 4B is another example of microbial response to antibiotic exposure using the base system, while also highlighting the fact that microbial metabolic activity may not recover following antimicrobial exposures. The compromised microbial health and metabolic activity levels that result from antimicrobial exposure can have significant detrimental impacts on real-world industrial processes which rely on stable, predictable, and/or desired microbial health and activity, such as in wastewater treatment plants. The insight gained here using the base system therefore demonstrates that changes in the microbial signals such as CO₂ production can act as a real-time early warning for events that negatively impact microbial health and activity, thereby serving as a “miner's canary” that can alert to changing water quality, and particularly the presence of toxic substances.

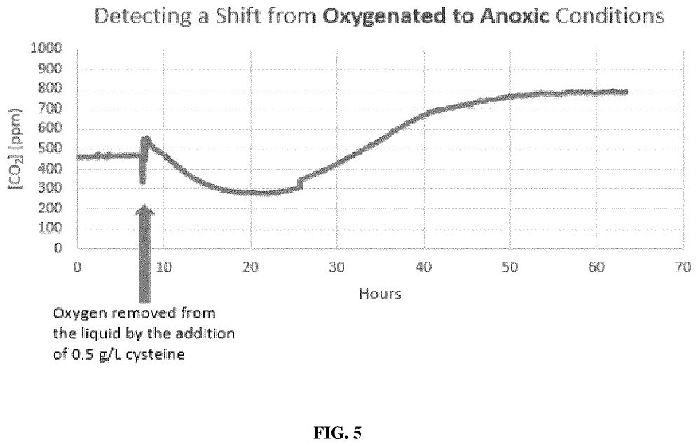

FIG. 5 presents data collected using the base system illustrating a rapid microbial response to changes in oxygen availability. In any aqueous environment, oxygen availability plays an important role in dictating microbial activity and overall water quality. This data demonstrates that microbial signals such as CO₂ production can be used to elucidate changes in oxygenation (e.g. a shift from oxygenated to anoxic environment) in natural and engineered systems. This is relevant for example in aeration tanks of wastewater treatment plants, where rapid changes in oxygenation can occur and lead to process upsets.

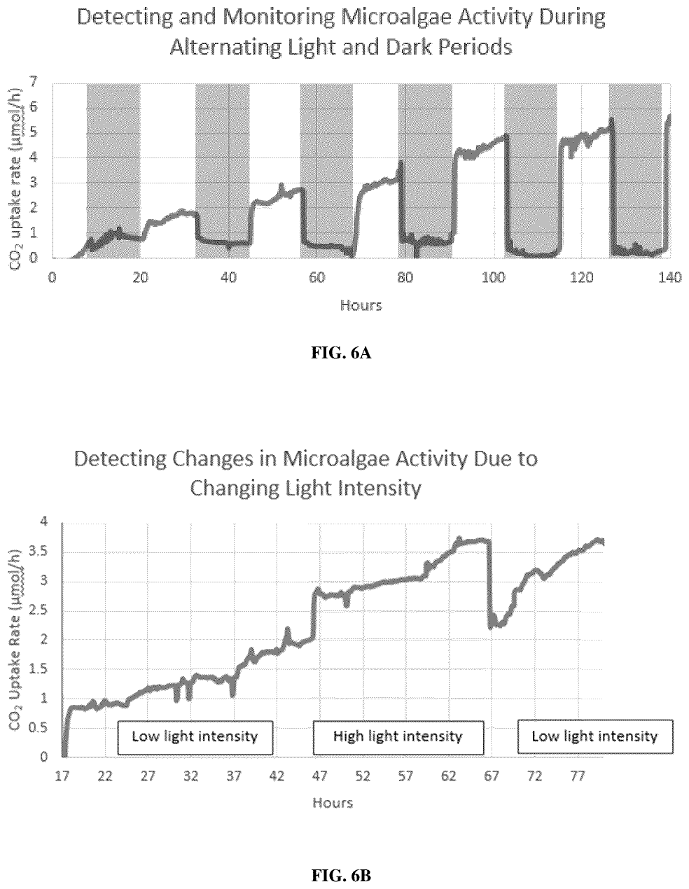

FIG. 6A and FIG. 6B presents data obtained from a slight modification of the base system by the inventors so that the consumption rather than production of microbial signals can be measured. The graphs in FIG. 6A and FIG. 6B illustrate the use of this modified system for the detection and monitoring of photoautotrophic microbes (e.g. microalgae). Microalgae are single-celled microbes which, given sufficient nutrients, can cause toxic algal blooms in rivers, lakes, and streams. In an industrial setting conversely, they offer many benefits in terms biofuel and pharmaceutical production, and as a source of numerous other value-added products. Like all plants, microalgae are photoautotrophs meaning they utilize light and CO₂ for energy and growth. By adjusting the composition of the sweeper gas used to carry microbial signals to the analyzer, such that it contains a known concentration of CO₂, we are able to detect and monitor microalgae activity by measuring the uptake in CO₂ caused by their presence and metabolism. The graph in FIG. 6A depicts microalgae activity, given as the rate of CO₂ consumed per hour, under alternating twelve-hour light-dark cycles. The grey boxes represent the dark periods. The graph in FIG. 6B depicts the effect of changing light intensity (i.e., changing from high to low then back to high light intensity) on microalgae activity.

The extensive laboratory-based research carried out with the base system and slight modifications thereof, as highlighted in each of the aforementioned graphs, provided important and necessary demonstration of fundamental and theoretical concepts related to microbial responses to intentional and controlled manipulation of microbial growth conditions. The tube within-a-tube design of the base system, whereby a microbial biofilm is cultivated inside a silicone tube that is housed within a larger diameter Tygon tube with sweeper gas flowing through the annular space and into a CO₂ analyzer, was sufficient for this fundamental research. However, the present disclosure is the result of comprehensive research and development by the inventors and is a non-obvious advancement of the base system that allows for the application of the scientific insights gained from the base system, such as to accomplish real-world (i.e. beyond just the laboratory setting) and real-time microbial monitoring in a range of environmental and industrial settings. Rather than observe microbial responses resulting from intentional and controlled manipulations of growth conditions of laboratory cultures, as in the use of the base system during laboratory-based research, the disclosed technology allows for monitoring the size, health, and metabolic activity level of native microbial populations in any aqueous environment. In situ monitoring of these populations, which can include pure cultures (axenic), mixed cultures (non-axenic), prokaryotes, eukaryotes, archaea, heterotrophs, autotrophs and mixotrophs, facilitates the use of microbial signal data to gain real-time insight into environmentally and industrially relevant systems, such as to solve numerous water and wastewater related issues.

In a further embodiment, a major modification to the base system whereby a membrane that is permeable to the microbial signal(s) of interest is used to collect microbial signals directly from the environment in which the membrane is placed. Note that herein “biomass scaffold” may be used to describe the permeable membrane and/or a rigid or flexible support to which the permeable membrane is affixed and/or incorporated within. The modification to the base system eliminates the need for any gas impermeable outer tube, nor does it require the formation of an annular space between a gas permeable inner tube and gas impermeable outer tube, as was required by the base system. To a large extent the published “enclosed” design of the base system (FIG. 1) was aimed at verifying the validity of using microbial signals as proxies for chemical parameters (e.g. nutrient concentration) in aqueous environments. This enclosed system design of FIG. 1 was necessary to set up mass balances as a very important verification step. The “non-enclosed” design of the disclosed technology represents a major novel and non-obvious advancement of the base system so that the concept of monitoring microbial signals can be used to report on local conditions in any aqueous environment.

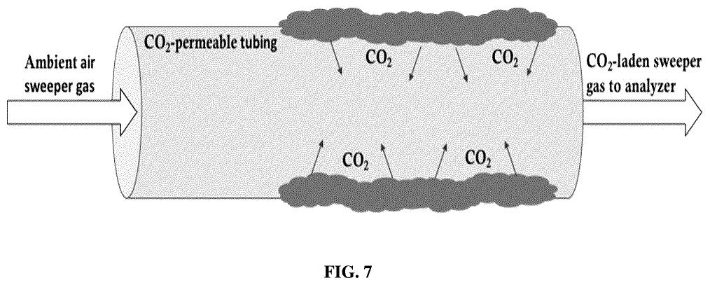

One further aspect of one embodiment of the disclosed technology is shown in FIG. 7. FIG. 7 is a schematic depicting a comprehensive modification of the base system whereby a permeable tubular membrane is used to grow biomass (e.g. biofilms) on the outer surface of the tube. According to FIG. 7, a permeable tubing (e.g. permeable to CO₂) can be used as the biomass scaffold, whereby microbial growth can occur on or near the outer surface of the tubing, allowing for the microbial signals of interest to diffuse through the tubing wall and into the lumen of the tube, where it can then be measured directly using an analyzer, or it may be shuttled via forced flow of a sweeper gas comprised of either ambient air or a specialty gas to a downstream analyzer (e.g. CO₂ analyzer).

There are numerous applications where such a microbial monitoring system can be of important and unique value. For example, the novel tool has been shown to distinguish sewage-induced spikes from baseline microbial activity. Further, software algorithms, machine learning, and artificial intelligence may be also be developed to determine site-specific thresholds for microbial signals and relevant alarm systems. Multiple microbial signals can also be measured simultaneously or in series with one another. In this way, a microbial “fingerprint” can be determined based on the unique combination of microbial signals produced in the monitored environment, which can offer improved and more specific insight into the chemical and physical conditions, as well as improved understanding of the microbial community composition. As well, future developments could combine the microbial signal data with other operational and environmental datasets to develop predictive models that can identify trends, patterns, associations, and interactions among parameters.

### Summary of Progress Towards Proposed Refinement

Several working prototypes of the disclosed technology have been developed and tested to demonstrate the sensitivity of the technology in the four general scenarios described above. Briefly, each embodiment involves the use of gas permeable membranes which may or may not be attached to or incorporated within a rigid or flexible structure (collectively referred to as a “biomass scaffold”), which is oriented in a manner that creates a gaseous cavity into or out of which microbial signals may diffuse but bulk water is excluded. The presence and concentration of microbial signals are detected and measured by suitable sensors, analyzers, and/or detectors which may be placed either immediately adjacent to the permeable membranes and/or at a distance from the membranes and housed within a “central hub”. Among many features, the central hub is capable of generating flow of a sweeper gas, logging data locally, transmitting data to cloud-based databases through telemetry, applying proprietary data algorithms, and alerting when custom or preset thresholds are exceeded.

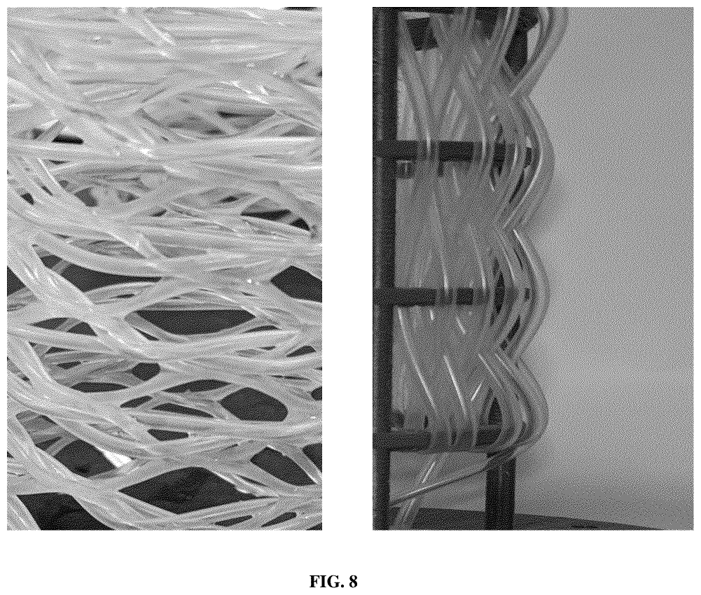

FIG. 8 is a diagram showing two iterations of the biomass scaffold prior their colonization with microbial biomass. The scaffold can take any geometrical shape to suit the desired application and could involve the use of a rigid or flexible frame to which permeable membrane material is attached, though the frame could also be excluded.

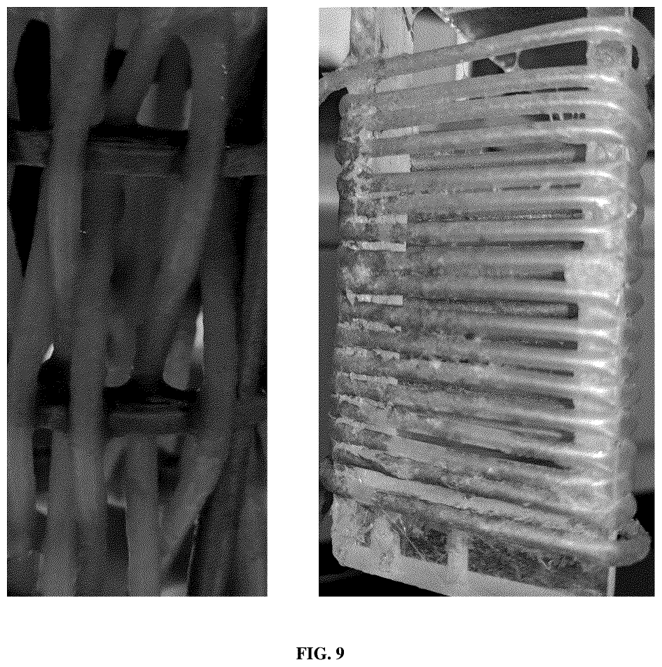

FIG. 9 is a diagram showing two iterations of the biomass scaffold after being colonized with microbial biomass. The production or consumption of microbial signals by the biomass growing on or near the scaffold can be determined in real-time and their concentration can be used to gain specific insights into the environments being monitored.

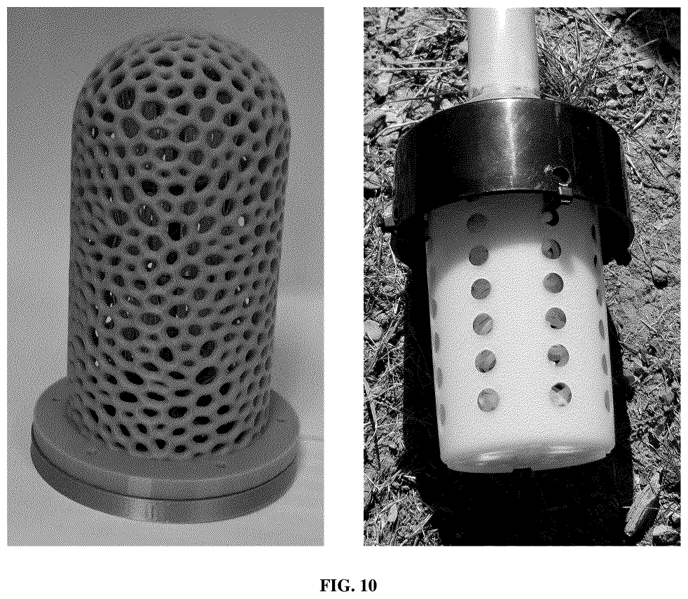

FIG. 10 is a diagram showing two iterations of the biomass scaffold with different protective casings around the scaffold. Use of a protective casing may be necessary to maintain the integrity of the scaffold during deployment in harsh or turbulent conditions, where physical damage may be possible.

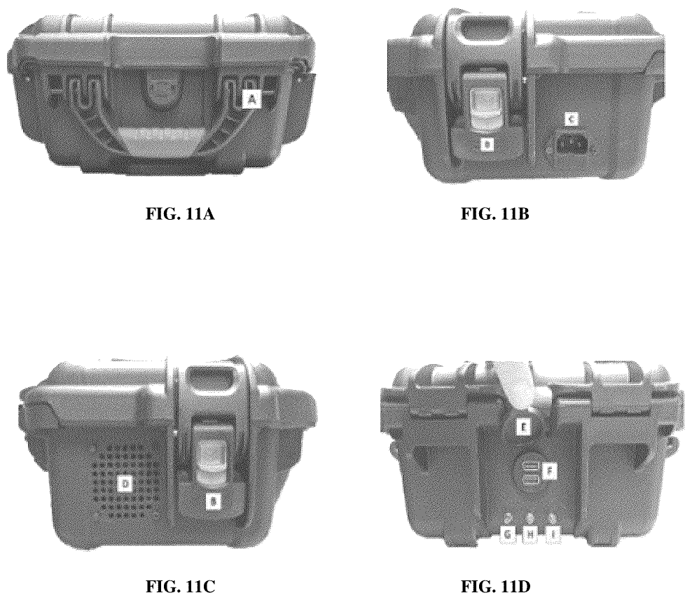

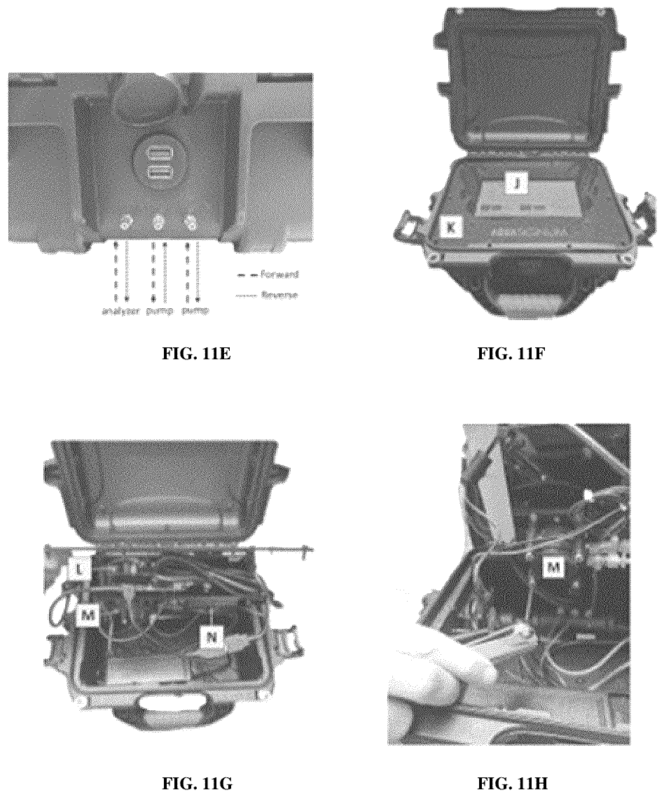

FIGS. 11A to 11H are labelled images of one embodiment of the central hub. FIG. 11A is a front view of the central hub. FIG. 11B is a right side view of the central hub. FIG. 11C is a left side view of the central hub. FIG. 11D is a rear view of the central hub. FIG. 11E is diagram illustrating direction of gas flow. FIG. 11F is a diagram of the central hub with the lid open. FIG. 11G is a diagram of the central hub with the lid lifted. FIG. 11H is a diagram of the central hub illustrating access of the internal pump.

In this embodiment and according to FIGS. 11A to 11H the central hub contains a Liquid Crystal Display (LCD) touchscreen J, as seen in FIG. 11F, for setting and adjusting operational settings such as flow rate of the sweeper gas, setting the data logging rate, allowing for calibration of the internal analyzer, and displaying microbial signal data.

According to FIGS. 11A to 11D, the central hub includes a soft grip handle A, a powerclaw latching system B, a power socket C, a cooling fan D, a cover for dual universal serial bus (USB) ports E, dual USB ports F, an analyzer gas port G that is connected to the gas analyzer, an outward gas port H that is outward in forward mode, an inward gas port I that is inward in forward mode. FIG. 11E illustrates the direction of gas flow where the dotted arrow indicates a forward direction and a solid arrow indicates a reverse direction.

According to FIG. 11G and FIG. 11H, the central hub also contains an internal peristaltic pump M used for generating flow of a sweeper gas though a connected biomass scaffold, as well as an internal CO₂ analyzer N and internal computer processing unit (CPU) L. The central hub can be connected to a local or global network to allow for real-time transmission of data to cloud-based databases or connected to existing process management systems such as supervisory control and data acquisition (SCADA) systems.

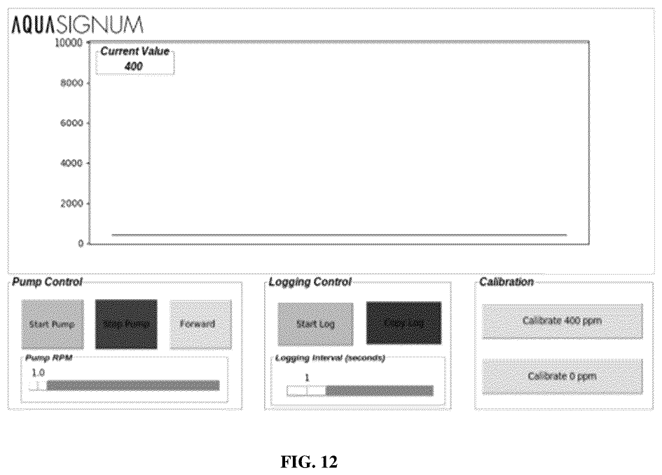

The central hub has proprietary software installed, which allows for coordinated processing such as pump control, real-time microbial signal measurement, data logging and transformation using set algorithms, maintaining network connection, and uploading data to cloud-based databases. FIG. 12 shows one embodiment of the software as displayed on the LCD touchscreen of the central hub. According to FIG. 12, the screenshot indicates the current microbial signal value and controls for Pump Control, Logging Control and Calibration.

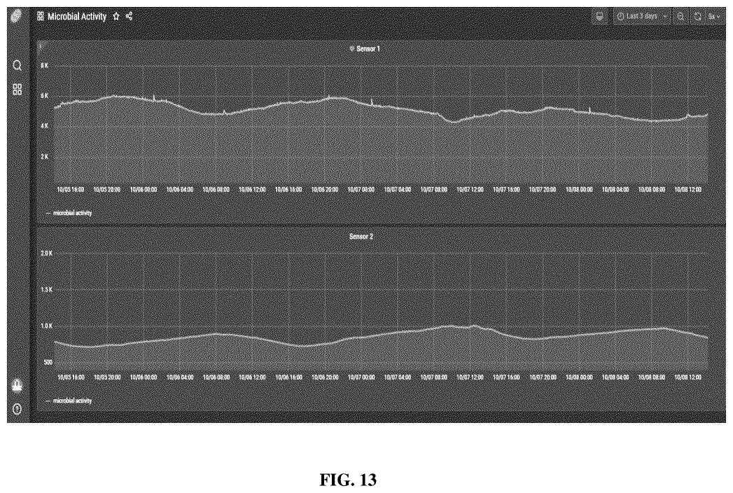

FIG. 13 is a diagram showing one embodiment of user-facing online dashboard used to display microbial activity data collected via the biomass scaffold and subsequently measured and transmitted to a cloud-based database by the central hub. The online dashboard can be fully configured to include built-in data algorithms as well as custom thresholds and alarm settings.

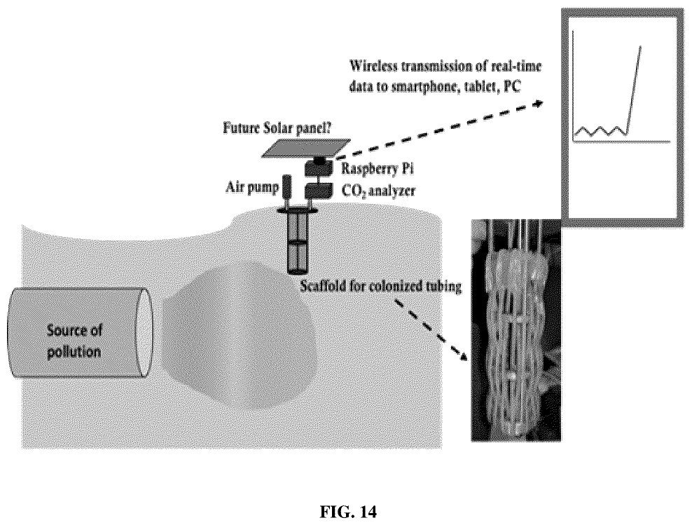

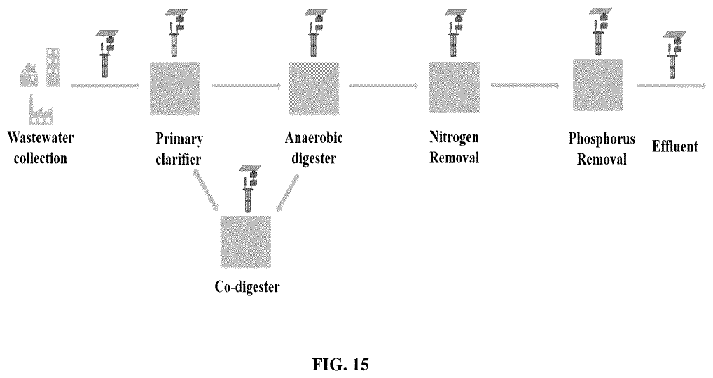

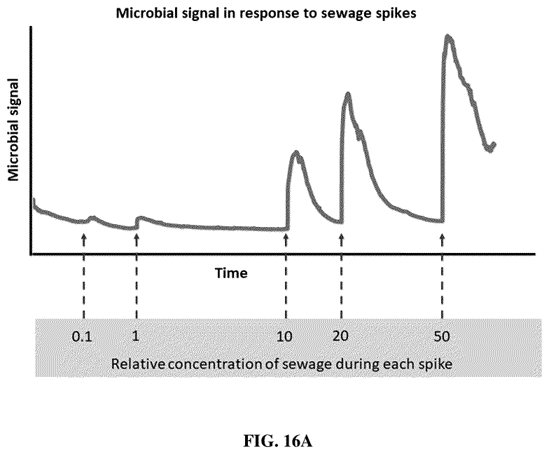

FIG. 14 is a schematic diagram showing one possible application of one embodiment of the disclosed technology. In this example, the microbial scaffold can be placed at or near the outfall of combined sanitary and storm sewer (“combined sewer pipe”) and used to provide real-time alerting to overflows and dumping of combined stormwater-sewer mixtures. This system pumps ambient air (or other sweeper gas) through a “biomass scaffold” comprised of gas (e.g. CO₂) permeable tubular membrane. This membrane is colonized with the native microbial population in the environment where it is placed, and becomes colonized as a biofilm attaches, grows, and develops indefinitely on the outer surface of the tube. FIG. 15 also shows the potential for a solar powered version of the technology, which would allow for deployment into remote regions where connections to an electricity grid are not possible. In addition to detecting when and where these sewage infiltration events occur, the magnitude of the measured change in microbial signals may also be used to infer the relative “strength” of the stormwater-sewage mixture (e.g. how much carbon is present or what the ratio of sewage to stormwater might be).

FIG. 15 is a schematic depicting the process steps of a generic wastewater treatment system. Given that wastewater treatment is critically dependent on stable, consistent, and predictable microbial activity, this diagram illustrates another possible application of one embodiment of the disclosed technology. In this example, the microbial scaffold can be placed in the wastewater influent, effluent, as well as in the tank(s) of any and/or all steps of the treatment process. Deployment of the technology in this fashion can provide wastewater treatment plant personnel with real-time insight into the microbial population size and/or microbial metabolic activity levels of the biomass carrying out the treatment process. Since “stable” or “baseline” plant operation differs significantly from plant to plant, the technology can help to elucidate and recognize stable operating conditions for individual plants, and can be used to provide early warning when chemical and/or physical parameters lead to deviation from stable operating conditions.

### Experimental Results

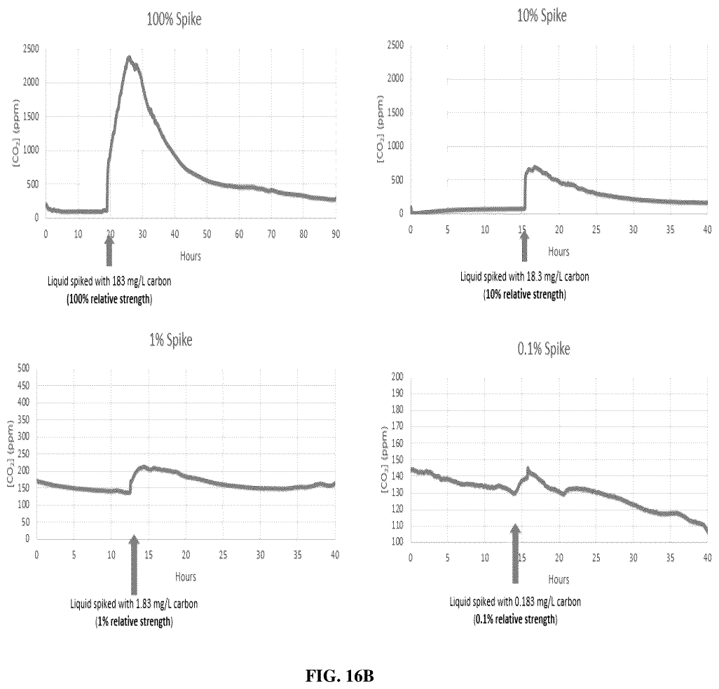

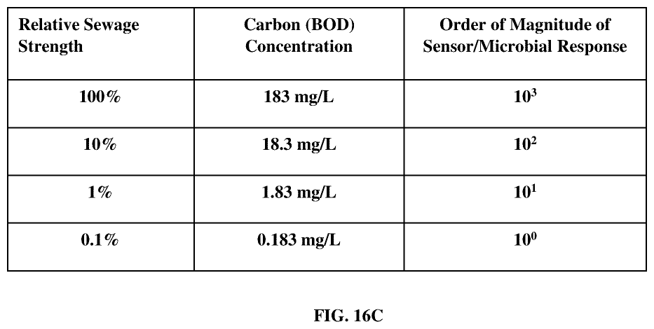

FIG. 16A is a graph illustrating microbial signal in response to simulated sewage spikes. According to FIG. 16A, real data is recorded by one embodiment of the technology and indicated here as a generic ‘microbial signal’. The microbial response to each sewage spike is further shown in the graphs depicted in FIG. 16B, in terms of real CO₂ production values. The aim of this dataset was to demonstrate the rapid and proportional microbial responses to changes in the chemical composition of the aqueous system relative to an established baseline, again validating the use of a microbial proxy for changes in chemical composition. FIG. 16C is a chart showing analysis of the data presented in FIG. 16B and indicates that indeed increasing and proportional microbial response can be expected with spikes of sewage with increasing strength.

It should be noted that the microbial response measured by the technology can in fact be a result that is desired by the customer, operator, or user etc. and does not necessarily have to be a proxy for any one chemical component. Rather, it can be used to ensure a healthy and active microbial population under a range of industrial (e.g. wastewater treatment, pharmaceutical, food and beverage, agriculture, etc.) or environmental (e.g. nutrient run-off detection, bioremediation detection) conditions.

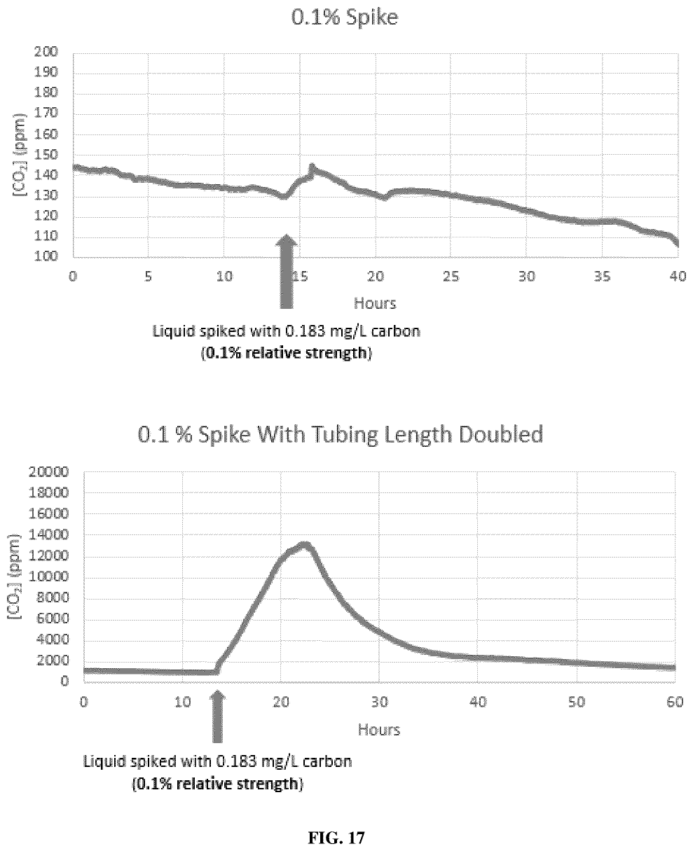

FIG. 17 are graphs showing the change in microbial response to sewage spikes when the length of the tubular membrane was doubled. FIG. 17 illustrates the ability to tailor the technology to desired applications, for example by increasing sensitivity to specific microbial signals of interest. Laboratory testing has shown that the sensitivity of the technology can be increased by increasing the length, surface area, or size of the colonizable permeable membrane (e.g. tubing).

As shown in FIG. 17, doubling the length of tubing in one embodiment of the technology led to a ten-fold increase in sensitivity. The length, surface area, or size of the permeable membrane (along with other parameters such as type and flow rate of sweeper gas) is customizable and can be tailored to specific applications and end-user needs. The potential for such modifications under control of the operator, especially adjustment of the sweeper gas flow rate, means that the disclosed technology can be used to monitor microbial signals over a very wide range of environmental conditions. That is, the strength and/or magnitude of the microbial signal can be dialed and/or tuned so that it falls within a desired range. The ability to adjust the sensitivity of the monitoring technology allows for monitoring in environments with very low levels of microbial signal as well as those with extremely high levels of microbial signal.

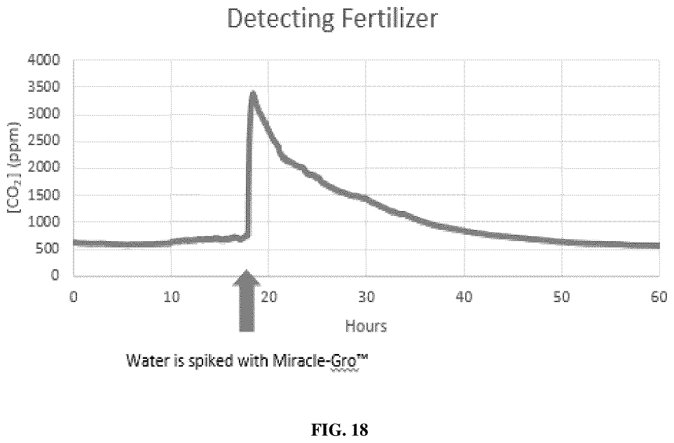

FIG. 18 is a graph illustrating the ability of the technology to detect changes in microbial signal caused by the presence of fertilizer in an aqueous environment. Nutrient run-off, particularly in the form of fertilizers from agricultural lands, is a major threat to water quality, can lead to eutrophication, and can greatly disrupt industries and ecosystems which depend on clean water. As seen in FIG. 18, experiments demonstrate that the disclosed technology effectively detects microbial responses to the presence of Miracle Gro™ fertilizer in water.

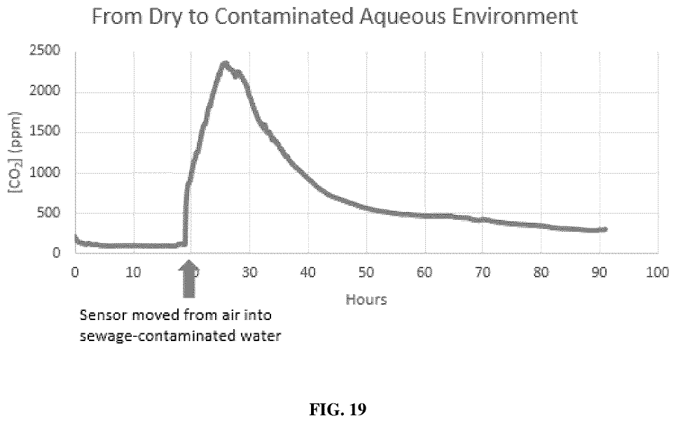

FIG. 19 is a graph illustrating the microbial response measured with one embodiment of the disclosed technology, whereby a membrane-bound biofilm was exposed to sewage after the biofilm had been dried out for many weeks. This demonstrated that when the membrane-bound biofilm is dried, it remains viable and highly sensitive to sewage constituents, and is able to produce a signal which is easily detected by the technology. The technology's tolerance to desiccation, as illustrated in FIG. 19, demonstrates the feasibility of using it in locations where conditions are intermittently wet and dry. This may include but is not limited to engineered systems such as storm water catchments, combined sewer-storm water pipes, other sewage infrastructure, and in natural environments such as beach sediments or intertidal zones.

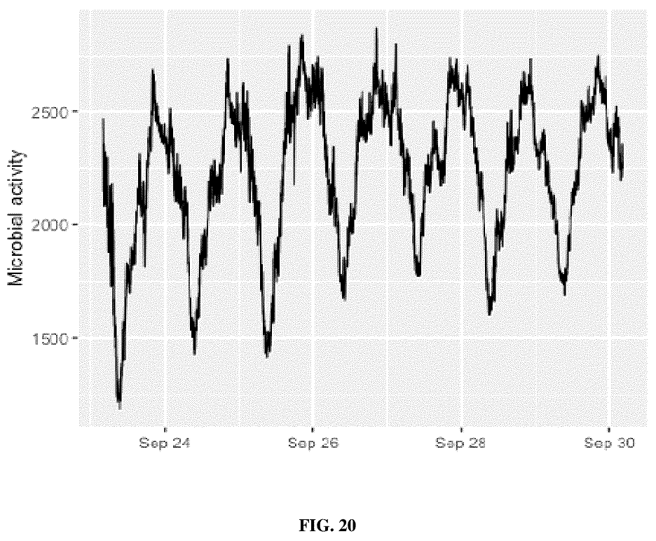

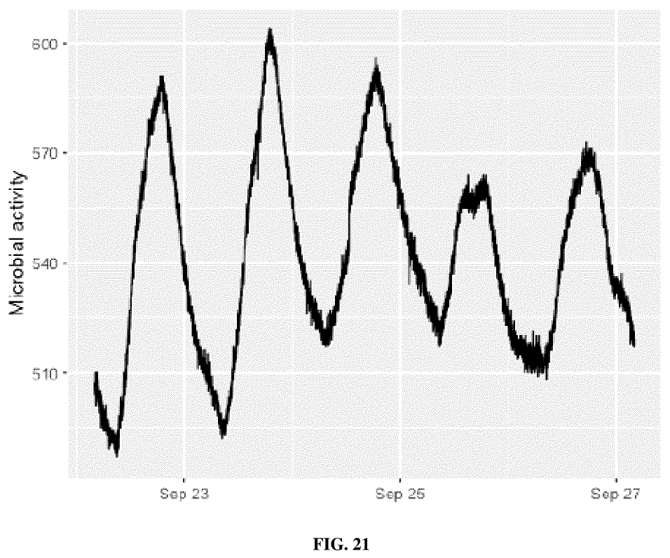

Real-world installations of the technology have provided unique insights into the stability of water quality in natural and engineered water and wastewater treatment systems. For example, FIG. 20 is a graph showing real-world (i.e. not laboratory) data collected by one embodiment of the technology when used to monitor the influent stream of a wastewater treatment plant. This graph demonstrates the ability of the technology to detect diurnal patterns in microbial activity. FIG. 21 is a graph showing real-world (i.e. not laboratory) data collected by one embodiment of the technology, from a natural stream system. Once again, the technology effectively detected a diurnal pattern in microbial activity occurring in the stream, this time with lower deviation intensity compared to the wastewater treatment plant influent.

### Further Developments

The current working prototype is one embodiment of the technology that utilizes a gas-permeable tubular membrane, which allows the diffusion of CO₂, which is used as the microbial signal. CO₂ produced by microbes colonizing the outside of the tubular membrane, or in the surrounding environment, diffuses into the lumen of the tube, where it travels to an analyzer via forced air flow. While this embodiment of the technology utilizes CO₂ as the microbial signal, it would be feasible to use other biologically-relevant gaseous compounds (e.g. CO, O₂, O₃, H₂, H₂S, CH₄, SO₂, N₂, NO₂, NO, N₂O etc.) as the microbial signal with only relatively minor changes to the technology. These changes could include selecting suitable sensors, analyzers, and/or detectors that are capable of measuring one or more signals of interest, and selecting a type of membrane that is permeable to said signals. A variety of different materials could be used to serve this purpose, including polymer or ceramic membranes consisting of polydimethylsiloxane (silicones), polyethylene, high-density polyethylene, low den si ty-polyethylene, polypropylene, polytetrafluoroethylene, fluorinated ethylene propylene, polyimide, polysulfone, cellulose acetate, perfluorosilicon, polyinethylpentene, poly(phenylene oxide), zeolite, aluminum oxide, silicon carbide, titanium dioxide, and zirconium dioxide.

The ability to use a variety of compounds beyond only CO₂ as the microbial signal greatly expands the potential applications of the novel technology. For example, measurement of N₂, NO₂, NO, and/or N₂O could be used to monitor microbial populations involved with nitrification, denitrification, and/or nitrogen cycling during wastewater treatment and/or during environmental bioremediation. Likewise, measurement of CH₄ could be used to monitor the efficiency of anaerobic digestion and subsequent biogas production during wastewater treatment or organic digestion. Measurement of O₂ could be used to monitor autotrophic microbial communities such as those used in microalgae-mediated biofuel and bioproduct production. In addition to being an important component of earth's stratosphere, O₃ has significant antimicrobial properties which are commonly exploited in both industrial and clinical settings to control and/or limit microbial growth. The ability to measure O₃ in such applications could therefore offer significant benefit in informing ozone exposure protocols. Measurement of H₂S could be used to detect microbes contributing to microbial induced corrosion. H₂S (along with SO₂) is also a minor product of microbial processes involved in wine making, and hence the ability to measure these compounds can be used for process monitoring and quality control therein. As a common microbial fermentation product, measurement of H₂ could also be used to monitor and evaluate industrial fermentation processes. Additionally, H₂, CO₂, and CO are used in microbial syngas fermentation, and the ability to measure these compounds can provide beneficial insights into syngas fermentation processes.

It is also possible to measure multiple of the aforementioned gaseous compounds in conjunction with one another. In this way, a “fingerprint” of the microbial population in any aqueous or non-aqueous natural or engineered environment can be elucidated. This in turn can allow for novel insights into microbially-mediated processes in natural and engineered systems, leading to significant beneficial outcomes.

Whereas the current embodiment of the technology involves the placement of permeable membranes into environments such that native microbial populations can be monitored, future embodiments can involve the monitoring of non-native microbes which can be attached to the membranes via a pre-treatment or a pre-colonization step prior to its placement into the environment. Such an approach could have significant utility in bioremediation projects such as to introduce, and subsequently monitor, the microbes added during bioaugmentation.

In addition to measuring the production of microbial signals, it would be feasible to also measure the consumption of microbial signals with only relatively minor changes to the technology. For example, CO₂ consumption, capture and/or sequestration by microbial populations (e.g. photoautotrophic and chemolithoautotrophic microbes) can be monitored by utilizing a sweeper gas with a consistent, known, and/or measurable concentration of CO₂. A comparison of the CO₂ concentration of the sweeper gas before and after its flow through the tubular membrane colonized on the outer surface by microbial biomass, can be used to determine the decrease, loss, or decline in signal caused by microbial consumption. Notably, this sweeper gas could be ambient air drawn from an outdoor location, since this air will contain a relatively consistent concentration of CO₂. The ability of this technology to monitor CO₂ consumption, capture and/or sequestration presents a significant benefit for example in algaculture and/or in monitoring for, and alerting to, the onset of algal blooms in natural and engineered water systems.

The following is a non-exhaustive list of use cases that have been identified in which application of this technology can offer beneficial outcomes:
Monitoring wastewater treatment process (each process step, influent, effluent)
Assessing “strength” of wastewater
Monitoring for and detecting nutrient “slug” in wastewater treatment plant influent
Monitoring for and detecting toxin(s) in wastewater treatment plant influent (providing early warning for toxic shock)
Informing and optimizing aeration in wastewater treatment
Informing and optimizing carbon addition in wastewater treatment
Monitoring wastewater treatment plant effluent to ensure treatment is complete
Monitoring wastewater treatment plant effluent to ensure ample chlorination/disinfection
Monitoring lagoon systems
Monitoring of anaerobic digester performance/biogas production (CO₂ or CH₄)
Monitoring for and detecting biofilm formation in pulp and paper systems
Monitoring for and detecting biofouling in water collection and distribution systems
Monitoring for and detecting biofouling in cooling towers
Monitoring for and detecting CO₂ uptake by photosynthetic microbes (e.g. microalgae)
Monitoring for and detecting eutrophication events and algal blooms
Monitoring for and detecting nutrient infiltration into receiving waters
Monitoring and evaluating bioremediation projects
Monitoring for and detecting microbial activity in wells and cisterns
Monitoring for and detecting microbial activity in hot water tanks (detecting conditions favourable for Legionella sp.)
Monitoring and optimizing fermentation processes
Monitoring and optimizing beer-brewing and wine-making processes
Monitoring and optimizing biofuel production (ethanol, methanol, etc.)
Monitoring for and detecting biofouling in ship ballasts
Monitoring and optimizing aquaculture processes
Monitoring for and detecting microbial activity in mine tailings ponds, dams, and dykes

The specific microbial signals that would be monitored in the aforementioned use cases depend on the specific conditions as well as the specific problems being addressed. The utility of the technology, along with the interpretation and analysis of signal data collected, could be enhanced by the addition of sensors, analyzers, and/or detectors capable of measuring relevant parameters besides the microbial signals themselves. These relevant parameters may include temperature, pH, dissolved oxygen, conductivity, redox, optical density, biological or chemical oxygen demand, total suspended solids, ATP content, phosphorus and its derivatives, nitrogen and its derivatives, humidity, moisture, and gas flow or liquid flow.

The current working prototype is one embodiment of the technology that involves the use of a Raspberry Pi computer within the central hub to control operating parameters (e.g. pump speed, data logging, network connectivity), although alternative processors could be used in future iterations. Further embodiments of this technology may include developing algorithms and computer and statistical models based microbial population size and/or microbial health and/or microbial metabolic activity data, which can be used to improve and inform both the operation of the technology itself and/or processes within the aqueous natural or engineered environments in which it is applied. Such algorithms as well as computer and statistical models may also be used to identify and recognize patterns and predict/forecast future events in the aqueous natural or engineered environments being monitored. Such algorithms and computer and statistical models may also be used either solely or in conjunction with additional testing, experimentation, and/or data to identify and expand upper and lower limits of detection of the technology under a range of environmentally and industrially relevant scenarios, as well as to set process-specific warning and alert thresholds of microbial populations and/or microbial health and/or microbial metabolic activity.

Further embodiments of the technology will include systems to improve the durability and robustness of the hardware for deployment in a range of potentially harsh and/or corrosive environments. These may include but are not limited to water and wastewater distribution and collection systems, anaerobic digesters, and other industrial processes.

Further embodiments may seek to integrate automation to sensor operation, as well as data acquisition, transmission, and storage to maintain ease of use.

Yet further embodiments may include improvements to back-end data processing as well as the user-facing application. There is also the potential for integration of machine learning and/or artificial intelligence into the data acquisition and analysis aspects of the technology.

In the present embodiment, CO₂ is measured and logged. It is then displayed on a graph and can be annotated, have thresholds, warnings, and alarms set, etc. However, integrating machine learning and artificial intelligence into the data analysis could allow faster detection and even prediction of microbially relevant events. Pattern recognition could be used to develop “fingerprinting” of microbial responses to various perturbations, such that response to one type of perturbation (e.g. change in CO₂ production rate caused by carbon addition) can be distinguished from another (e.g. change in CO₂ production rate caused by nitrogen or phosphorous addition). This “fingerprinting” can be expanded by measuring multiple microbial signals in series.

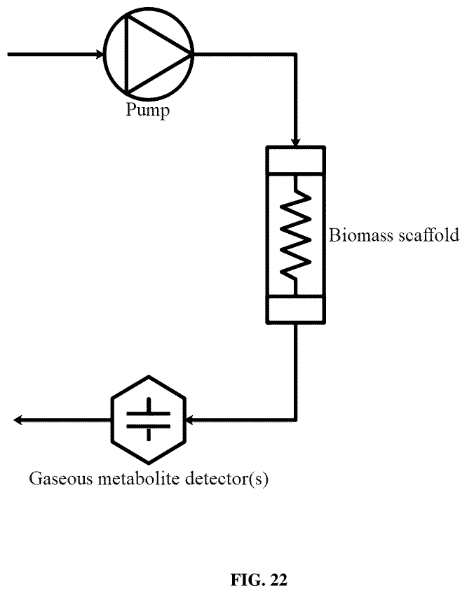

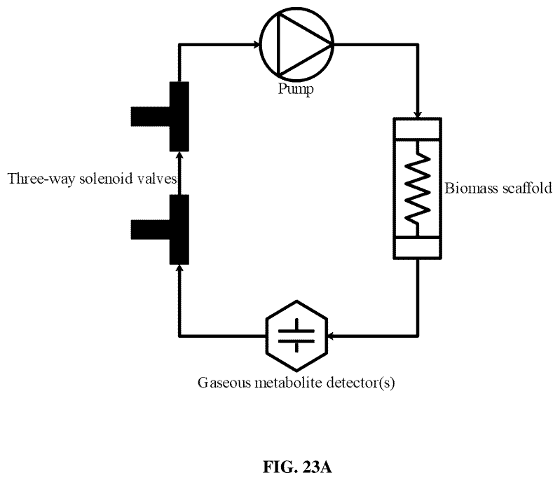

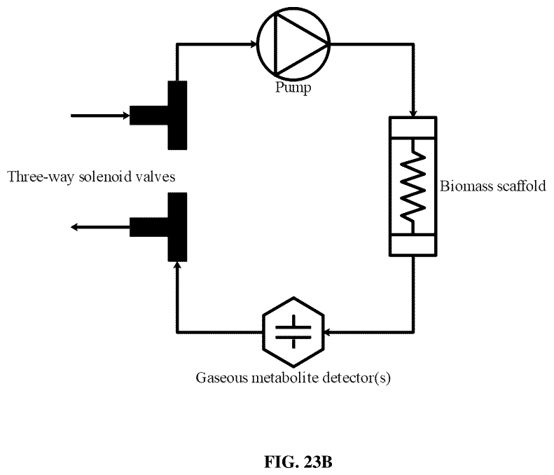

Further embodiments can include solenoid or other valves to interrupt and/or direct the flow of sweeper gas, for example, to shuttle the gas to alternative analyzers or to allow for interchange between various configurations of the technology. FIG. 22 is a schematic diagram showing the “open-loop” configuration of the technology whereby the sweeper gas flows in a once-through linear direction. FIG. 23A is a schematic diagram showing the “closed-loop” configuration of the technology whereby the sweeper gas is cycled continuously through the system. FIG. 23B is a schematic diagram showing the use of solenoid valves to exchange between the “closed-loop” and “open-loop” configurations of the technology.

While the current working prototype is one embodiment of the technology whereby the sweeper gas flows in the “open-loop” configuration, as depicted by the schematic in FIG. 22, use of solenoid or other valves can offer the ability operate the technology in a “closed-loop” configuration as depicted in the schematics shown in FIG. 23A and FIG. 23B. Operation in closed-loop configuration can be used to increase the sensitivity of detection by allowing for amplification of the microbial signals which may be advantageous in systems exhibiting low microbial activity.

While various inventive implementations have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive implementations described herein. More generally, those skilled in the art will readily appreciate that all parameters and configurations described herein are meant to be exemplary inventive features and that other equivalents to the specific inventive implementations described herein may be realized. It is, therefore, to be understood that the foregoing implementations are presented by way of example and that, within the scope of the appended claims and equivalents thereto, inventive implementations may be practiced otherwise than as specifically described and claimed. Inventive implementations of the present disclosure are directed to each individual feature, system, article, and/or method described herein. In addition, any combination of two or more such features, systems, articles, and/or methods, if such features, systems, articles, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, implementations may be constructed in which acts are performed in an order different than illustrated, which may include performing some acts simultaneously, even though shown as sequential acts in illustrative implementations.

All definitions, as defined and used herein, should be understood to control over dictionary definitions, definitions in documents incorporated by reference, and/or ordinary meanings of the defined terms.

1. A method of real-time monitoring of microbial signals in aqueous environments using a sensor monitoring system, the method comprising:
placing one or more membranes with permeability to one or more signals of interest into aqueous environments, such that a gaseous cavity is formed and into or out of which microbial signals is diffusible but bulk water is excluded;
collecting one or more microbial signals of interest crossing the membrane via diffusion and whose presence and concentrations are representative of the population size, health, or metabolic activity level of microbes growing on or near the permeable membranes;
passing the microbial signals of interest to sensors, analyzers, or detectors capable of measuring the presence and concentrations of the microbial signals of interest; and
analyzing microbial signal data to provide information about the aqueous environments being monitored.

2. The method of claim 1 wherein the microbial signals are gaseous compounds that are produced or consumed during microbial growth or used as a metabolite during microbial metabolic activity.

3. The method of claim 2 wherein the gaseous compounds are selected from a list consisting of CO₂, CO, O₂, O₃, H₂, H₂S, CH₄, SO₂, N₂, NO₂, NO, and N₂O.

4. The method of claim 1 wherein one or more suitable membranes are paired with one or more suitable sensors, analyzers, or detectors to allow for monitoring of one or more specific microbial signals of interest.

5. The method of claim 1 wherein the signals are produced by microbes that are native to the environment that is being monitored.

6. The method of claim 1 wherein the signals are produced by microbes that are not native to the environment being monitored but are attached to the membrane via a pre-treatment or a pre-colonization step prior to placing the membrane into the environment that is being monitored.

7. The method of claim 1 wherein the microbes being monitored is selected from a list consisting of pure cultures (axenic) or mixed cultures (non-axenic), prokaryotes, eukaryotes, archaea, heterotrophs, autotrophs and mixotrophs.

8. The method of claim 1 wherein the membranes are tubes or sheets which are attached to a rigid or flexible frame.

9. The method of claim 1 wherein the signals are measured by sensors, analyzers, or detectors placed near or adjacent to the permeable membrane, such that the signals can be passively measured without requiring forced air flow.

10. The method of claim 1 wherein the signals are measured by sensors, analyzers, or detectors placed at a distance from the permeable membrane, such that the signals are channeled to downstream sensors, analyzers, or detectors at known, measurable, or controllable flow rates using a sweeper gas whose flow may be generated by pumps, vacuums, compressed air or compressed gas.

11. The method of claim 10 wherein the signals are carried to downstream sensors, analyzers, or detectors via forced air flow using ambient air as the sweeper gas.

12. The method of claim 10 wherein the signals are carried to downstream sensors, analyzers, or detectors via forced air flow using a specialty non-air gas as the sweeper gas, such that the exact composition of the sweeper gas can be known or controlled.

13. The method of claim 10 wherein the sweeper gas follows a once-through “open-loop” flow path, or continuously cycled through a “closed-loop” flow path to allow for accumulation of microbial signals.

14. The method of claim 10 wherein modifications to parameters such as the surface area of the membrane or flow rate of the sweeper gas may be used to adjust the sensitivity of the system to accommodate monitoring of environments with microbial signals ranging from very low to very high levels.

15. The method of claim 1 wherein the microbial signal data is subjected to interpretation or analyses which includes triggering threshold alarms, and informing data algorithms for pattern recognition, machine learning, automation, or artificial intelligence.

16. The method of claim 1 wherein microbial signal data is incorporated into existing data management systems, supervisory control and data acquisition (SCADA) systems to inform or control system processes.

17. The method of claim 1 wherein microbial signal data is measured in wastewater collection and treatment systems, or septic tanks, to provide data selected from a list consisting of detection of toxins or nutrient shock loads, a proxy measurement for biological oxygen demand and its removal during treatment, feedback related to system parameters such as aeration and/or chemical dosing, and monitoring of effluent to ensure adequate and/or optimal treatment has occurred.

18. The method of claim 1 wherein microbial signal data is measured in natural water systems and used to detect environmentally and ecologically harmful events selected from a list consisting of sewage infiltration, nutrient pollution, and algal blooms.

19. The method of claim 1 wherein microbial signal data is used to provide alerting to the onset and severity of unanticipated or unwanted microbial growth and used to inform biocide and other antimicrobial dosing protocols, within structures selected from a list consisting of drinking water collection, treatment, and distribution systems, wells and cisterns, cooling towers, and industrial processes requiring and relying on sterility or controlled microbial growth.

20. The method of claim 1 wherein microbial signal data is used to monitor and evaluate system performance, inform system operations and modifications therein, and alert to system upsets, within structures selected from a list consisting of fermentation, biogas production, bioprocessing, and bioremediation systems.

21-46. (canceled)
